(19) United States
(12) Patent Application Publication
(10) Pub. No.: US 2014/0275867 A1
(43) Pub. Date: Sep. 18, 2014

(54) METHODS AND SYSTEMS FOR SHAPING DRIVE PULSES IN A MEDICAL DEVICE

(71) Applicant: COVIDIEN LP, Mansfield, MA (US)

(72) Inventors: Chris J. Meehan, Golden, CO (US); Daniel Lisogurski, Boulder, CO (US)

(73) Assignee: Covidien LP, Mansfield, MA (US)

(21) Appl. No.: 13/842,118

(22) Filed: Mar. 15, 2013

(51) Int. Cl. A61B 5/00; A61B 5/024

(52) U.S. Cl. CPC A61B 5/72; A61B 5/02416
USPC 600/310

(57) ABSTRACT

Systems and methods are provided for shaping drive pulses in a medical device. In some embodiments, signal channel characteristics introduce undesirable channel effects including signal distortions such as droop in a square wave pulse. In some embodiments, the system may shape light drive pulses to compensate for channel effects. Light drive characteristics may be determined based on, for example, modeling of components and/or iterative calibration techniques. The output of the channel may be used to determine physiological information such as blood oxygen saturation and respiration rate.

The present disclosure relates to processing physiological signals, and more particularly, to shaping drive pulses to compensate for signal channel characteristics.

### SUMMARY

Methods and systems are provided for shaping light drive pulses in a medical device such as a pulse oximeter. In some embodiments, a signal channel characteristic of a signal channel may be determined. The signal channel may include the components, interconnects, and other paths through which a signal travels. The signal channel characteristic may include phase shifts, additive noise, frequency distortions, amplitude distortions, and other modifications to a signal passing through the channel. In some embodiments, light drive pulses input to a channel may be shaped, such that, for example, the convolution of the light drive pulse and the channel characteristics results in an output signal with a desired shape. In an example, a high pass filter of a channel, used to attenuate ambient light from a detected signal, may also add droop to a square wave pulse. To counteract the droop or other channel characteristics, the light drive pulse may be shaped such that the channel characteristics and the pulse shaping combine to produce an output signal with a desired shape or characteristics.

In some embodiments, channel characteristics are determined by modeling of the signal channel components. In some embodiments, channel characteristics are determined by analyzing an output signal and updating light drive parameters based on the analysis, for example, in an iterative process. In some embodiments, a combination of modeling and output analysis is used.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal including a red light drive pulse and an IR light drive pulse in accordance with some embodiments of the present disclosure;

FIG. 2B shows an illustrative plot of a detector signal that may be generated by a sensor in accordance with some embodiments of the present disclosure;

FIG. 3 is a perspective view of an embodiment of a physiological monitoring system in accordance with some embodiments of the present disclosure;

FIG. 4 shows an illustrative signal processing system in accordance with an embodiment that may implement the signal processing techniques described herein;

FIG. 5 is a block diagram of an optical monitoring system in accordance with some embodiments of the present disclosure;

FIG. 6 shows an illustrative determination of pulse shaping, in accordance with some embodiments of the present disclosure;

FIG. 7 shows an illustrative signal chain including a test subject in accordance with some embodiments of the present disclosure;

FIG. 8 shows an illustrative plot of system signals in accordance with some embodiments of the present disclosure; and

FIG. 9 is a flow diagram showing illustrative steps for shaping drive pulses in accordance with some embodiments of the present disclosure.

### DETAILED DESCRIPTION OF THE FIGURES

The present disclosure is directed towards shaping drive pulses in a physiological monitoring system. A physiological monitoring system may monitor one or more physiological parameters of a patient, typically using one or more physiological sensors. For example, the physiological monitoring system may include a photoplethysmograph for pulse oximetry or other suitable monitoring. Signals may pass through a signal channel in addition to interacting with a test subject, and the output of the signal to be analyzed may include both physiological signal responses and channel signal responses. In some embodiments, the channel response may include one or more channel effects that correspond to undesirable signal features. In some embodiments, shaping of the input signal may be used to mitigate the impact of the channel response on the physiological parameters determined from the output signal. As used herein, mitigation of channel effects may include attenuating, correcting, reducing, counteracting, or any other suitable changes corresponding to altering the presence of some or all of one or more signal features, such as features in the channel response. In some embodiments, mitigating channel effects may enhance determination of physiological parameters. In some embodiments, shaping the input signal may be a computationally efficient technique because it does not require processing or filtering of every output signal to remove channel response features, but rather can be based on an intermittent computation.

In some embodiments, distortion, noise, and other signal features are defined in relation to a channel. The channel may include any suitable elements in the path between the transmitter and receiver or digitized signal. As used herein, the channel is not considered to include the test subject, such that channel effects can be mitigated and the quality of determining physiological parameters improved by, for example, input signal shaping. The channel may include, for example, electrical components such as digital to analog converters, current sources, LEDs, photo-detectors, trans-impedance amplifiers, passive and active filters and analog to digital converters, any other suitable components, or any combination thereof.

In the case of a pulse oximeter, or other devices utilizing a light signal, the channel may include any suitable elements of light drive signal generation, light drive signal transmission, light drive signal amplification, light drive signal filtering, light signal generation, light signal transmission, light signal filtering, light signal detection, detected light signal transmission, detected light signal amplification, detected light signal filtering, any other suitable processing, filtering, amplification, or any combination thereof. In the example illustrated in FIG. 5 below, the channel includes transmission of a light drive signal, a light emitter, a light detector, and light signal receiving circuitry.

In some embodiments where the signal interacts with a test subject, the signal may pass through a first portion of the channel, followed by the test subject, followed by a section portion of the channel. Thus, it will be understood that elements of the channel need not be contiguous. It will be understood that any suitable elements may be included or excluded from the channel as it is considered herein. For example, the channel may include transmission and generation of a light signal, but need not include generation of the light drive signal.

In some embodiments, channel effects of the channel response may include undesired signal features caused by, for example, channel components. In some embodiments, components of the channel are non-ideal, in that they may add noise, gain, phase shifts, droop, overshoot, undershoot, slow rise time, slow fall time, frequency clipping, ringing, waveform shaping, other undesirable features, or any combination thereof. In some embodiments, light drive shaping of light pulses may be to mitigate the presence of these undesired features in the received signal. In an example where a square light pulse passes through a channel, the low pass effect of the channel may slow the rise time of the pulse. For example, a high pass filter used to attenuate ambient light may cause undesired droop and/or phase shifts in a square wave signal. In another example, the response rate of a photodetector may cause undershoot in detection of a square light pulse. In another example, the frequency response of an analog-to-digital converter that receives a square wave may cause signal errors. In another example, the received light pulse may not immediately return to zero when a light drive signal is shut off. Thus, the resulting waveform in any or all of these examples may be filtered, attenuated, phase shifted and spread out in time due to the response of the channel.

In some embodiments, phase shifting may occur when square light pulses pass through the channel. A square pulse may be represented by a wide frequency spectrum of sinusoids. The channel may impose varying amounts of droop, rise and fall time, and other distortions on each respective frequency, which may result in a spreading of a square pulse.

In some embodiments, the shape of a light drive pulse may be shaped such that the output of the channel substantially includes a desired waveform. For example, the convolution of the channel response and a shaped input signal may result in a desired output characteristic, such as a square wave. For example, if the detected signal in response to a square wave droops over the duration of a square wave pulse, the drive pulse may include increased amplitude over the course of the pulse to compensate for the droop. As used herein, pulse shaping may alternatively be referred to as pre-shaping because it is applied to the pulse before generation of the light signal based on predetermined parameters.

In the case of a photoplethysmograph, the signal may be a light signal partially attenuated by a subject's tissue. The attenuation or other signal characteristics associated with the subject's tissue may be the desired portion of the signal that is being measured by the oximeter. Pulse shaping may be configured to counteract or mitigate the undesired channel response on the signal, while maintaining the desired physiological response of the signal. For example, a physiological response may correspond primarily to amplitude attenuation of a signal. The channel response may include the undesirable signal feature of phase shifting. In some embodiments, the channel may be modeled and the phase shifts mitigated by pulse shaping, without significantly altering the contribution by the subject's tissue from the information contained in the signal.

In some embodiments, components of the channel that contribute to undesirable channel effects may be predictable based on the design, implementation, components present, or other suitable information. A channel response may be determined by modeling components, by training using calibration signals, by iteratively refining pulse shaping characteristics, by refining a manufacturing calibration based on further calibration or modeling, by any other suitable technique, or any combination thereof. In some embodiments, iterative refining of pulse shaping may include iteratively changing the shape of an input pulse until the desired characteristics of the output are achieved.

In some embodiments, shaping of a light signal may be a computationally efficient technique as compared to filtering of an output signal to mitigate channel effects. In some embodiments, the presence of undesirable channel effects is relatively constant, and thus can be determined significantly less frequently than physiological parameters are determined. The determined shaping may be stored in memory and retrieved for generating light drive pulses, for example using a digital output passed to a digital-to-analog converter. In some embodiments, the system may determine a pulse shaping at system turn-on, or when a new probe is added to the device. In another embodiment, intermittent calibrations may be required to determine pulse shaping after a particular interval of time (e.g., once a day), after a particular amount of use (e.g., every 100 hours), at any other suitable time, or any combination thereof. In some embodiments, pulse shaping reduces power consumption due to the reduced processor load as compared to filtering of an output signal.

In some embodiments, the channel effects of a particular probe may be considered in determining shaping parameters by the system recognizing the particular probe and retrieving predetermined stored parameters. Removable components such probes, sensors or patient cables can be identified by the system, for example by sending known signals to different pins, by a resistor ID, or by an embedded non-volatile memory. In some embodiments, the monitor may include a selectable list or menu including, for example, model numbers associated with cable assemblies, probes, and other components.

In some embodiments, pulse shaping may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate, respiration rate, respiration effort, blood pressure, any other suitable parameter, or any combination thereof. Exemplary embodiments of determining respiration rate are disclosed in Addison et al. U.S. Patent Publication No. 2011/0071406, published Mar. 24, 2011, which is hereby incorporated by reference herein in its entirety. Exemplary embodiments of determining respiration effort are disclosed in Addison et al. U.S. Patent Publication No. 2011/0004081, published Jan. 6, 2011, which is hereby incorporated by reference herein in its entirety. Exemplary embodiments of determining blood pressure are disclosed in Addison et al. U.S. Patent Publication No. 2011/0028854, published Feb. 3, 2011, which is hereby incorporated by reference herein in its entirety. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is selected to be of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to determine ambient signal levels, reduce overlap of the light drive pulses, allow time for light sources to stabilize, reduce heating effects, reduce power consumption, for any other suitable reason, or any combination thereof.

It will be understood that the pulse shaping techniques described herein are not limited to pulse oximeters and may be applied to any suitable medical and non-medical devices. For example, the system may include probes for regional saturation (rSO₂), respiration rate, respiration effort, continuous non-invasive blood pressure, saturation pattern detection, fluid responsiveness, cardiac output, any other suitable clinical parameter, or any combination thereof.

The following description and accompanying FIGS. 1-9 provide additional details and features of some embodiments of shaping drive pulses in a medical device. It will be understood that while many of the examples described herein are directed to the shaping of square wave signals, any suitable pulse shaping with any suitable waveforms may be used. For example, the system may use pulse shaping with sinusoidal waves, triangle waves, sawtooth waves, pulse width modulated signals, any other suitable signals, or any combination thereof. It will be understood that some waveforms such as sinusoids may not have an “off” period as described for square light pulses, but rather may have portions during which relatively more and less light is emitted. It will also be understood that drive pulse shaping may be used in combination with other techniques for improving system performance and the detection of physiological parameters, such as equalization, filtering, drive pulse modulation, the use of multiple wavelengths of light, digital processing, analog processing, any other suitable techniques, or any combination thereof.

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter. In some embodiments, all or some of sensor 102, monitor 104, or both, may be referred to collectively as processing equipment.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate physiological signals. In one embodiment, the red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

Light drive circuitry 120, as discussed above, may be configured to generate a light drive signal that is provided to light source 130 of sensor 102. In some embodiments, light drive circuitry 120 may shape or modify waveform pulses based on input from control circuitry 110, back end processing circuitry 170, user input, any other suitable input, or any combination thereof. For example, the waveform may be shaped with the inverse of square wave droop, such that droop effects added to the signal by front end processing high pass filtering is cancelled and the resulting wave is substantially square. In some embodiments, pulse shaping may be performed by light drive circuitry 120, control circuitry 110, any other suitable circuitry, or any combination thereof. For example, light drive circuitry 110 may shape pulses based on information from back end processing circuitry 170.

The light drive signal may, for example, control the intensity of light source 130 and the timing of when light source 130 is turned on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. As will be described in detail below, these pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red drive pulse 202 may have a higher amplitude than IR light drive pulse 204 because red LEDs may be less efficient than IR LEDs at converting electrical energy into light energy. In some embodiments, the output levels may be the equal, may be adjusted for nonlinearity of emitters, may be modulated in any other suitable technique, or any combination thereof. Additionally, red light may be absorbed and scattered more than IR light when passing through perfused tissue. When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary any that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark during that period.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of a detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current waveform 214 may be generated in response to a light source being driven by the light drive signal of FIG. 2A. The valleys of detector current waveform 214 may be synchronous with periods of time during which no light is being emitted by the light source. While no light is being emitted by a light source during the valleys, detector current waveform 214 may not fall all of the way to zero.

It will be understood that detector current waveform 214 may be a partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current or other signal associated with the received light as similar as possible to detector current waveform 214.

Referring back to FIG. 1, front end processing circuitry 150, which may receive a detection signal, such as detector current waveform 214, may include analog conditioning 152, demultiplexer 154, digital conditioning 156, analog-to-digital converter (ADC) 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 158, which may convert the conditioned analog signal into a digital signal. Analog-to-digital converter 158 may operate under the control of control circuitry 110. Analog-to-digital converter 158 may use timing control signals from control circuitry 110 to determine when to sample the analog signal. Analog-to-digital converter 158 may be any suitable type of analog-to-digital converter of sufficient resolution to enable a physiological monitor to accurately determine physiological parameters.

Demultiplexer 154 may operate on the analog or digital form of the detector signal to separate out different components of the signal. For example, detector current waveform 214 of FIG. 2B includes a Red component, an IR component, and at least one ambient component. Demultiplexer 154 may operate on detector current waveform 214 of FIG. 2B to generate a Red signal, an IR signal, a first ambient signal (e.g., corresponding to the ambient component that occurs immediately after the Red component), and a second ambient signal (e.g., corresponding to the ambient component that occurs immediately after the IR component). Demultiplexer 154 may operate under the control of control circuitry 110. For example, demultiplexer 154 may use timing control signals from control circuitry 110 to identify and separate out the different components of the detector signal.

Digital conditioning 156 may perform any suitable digital conditioning of the detector signal. Digital conditioning 156 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal. A particular embodiment of ambient subtraction using a high pass filter is disclosed in co-pending U.S. application Ser. No. 13/484,808, filed May 31, 2012, entitled “OPTICAL INSTRUMENT WITH AMBIENT LIGHT REMOVAL,” which is hereby incorporated by reference herein in its entirety.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 158. This may be achieved by applying gain to the detection signal by analog conditioning 152 to map the expected range of the detection signal to the full or close to full output range of analog-to-digital converter 158. The output value of analog-to-digital converter 158, as a function of the total analog gain applied to the detection signal, may be given as:

ADC Value=Total Analog Gain×[Ambient Light+LED Light]

Ideally, when ambient light is zero and when the light source is off, the analog-to-digital converter 158 will read just above the minimum input value. When the light source is on, the total analog gain may be set such that the output of analog-to-digital converter 158 may read close to the full scale of analog-to-digital converter 158 without saturating. This may allow the full dynamic range of analog-to-digital converter 158 to be used for representing the detection signal, thereby increasing the resolution of the converted signal. In some embodiments, the total analog gain may be reduced by a small amount so that small changes in the light level incident on the detector do not cause saturation of analog-to-digital converter 158.

However, if the contribution of ambient light is large relative to the contribution of light from a light source, the total analog gain applied to the detection current may need to be reduced to avoid saturating analog-to-digital converter 158. When the analog gain is reduced, the portion of the signal corresponding to the light source may map to a smaller number of analog-to-digital conversion bits. Thus, more ambient light noise in the input of analog-to-digital converter 158 may results in fewer bits of resolution for the portion of the signal from the light source. This may have a detrimental effect on the signal-to-noise ratio of the detection signal. Accordingly, passive or active filtering (e.g., high pass filtering) or signal modification techniques may be employed to reduce the effect of ambient light on the detection signal that is applied to analog-to-digital converter 158, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further physiological signals received from front end processing circuitry 150. For example, processor 172 may determine one or more physiological parameters based on the received physiological signals. Memory 174 may include any suitable computer-readable media capable of storing information that can be interpreted by processor 172. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with use interface 180 and communication interface 190.

User interface 180 may include user input 182, display 184, and speaker 186. User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth. In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate information, blood pressure, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected to monitor 314 (not shown). Monitor 314 may be configured to calculate physiological parameters based at least in part on data relating to light emission and acoustic detection received from one or more sensor units such as sensor unit 312. For example, monitor 314 may be configured to determine pulse rate, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. Cable 324 may connect to monitor 314 at port 336. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, all or some of monitor 314 and multi-parameter physiological monitor 326 may be referred to collectively as processing equipment.

FIG. 4 shows illustrative signal processing system 400 in accordance with an embodiment that may implement the signal processing techniques described herein. Signal processing system 400 includes input signal generator 410, processor 412 and output 414. In the illustrated embodiment, input signal generator 410 may include pre-processor 420 coupled to sensor 418. As illustrated, input signal generator 410 generates an input signal 416. In some embodiments, input signal 416 may include one or more intensity signals based on a detector output. In some embodiments, pre-processor 420 may be an oximeter and input signal 416 may be a PPG signal. In an embodiment, pre-processor 420 may be any suitable signal processing device and input signal 416 may include PPG signals and one or more other physiological signals, such as an electrocardiogram (ECG) signal. It will be understood that input signal generator 410 may include any suitable signal source, signal generating data, signal generating equipment, or any combination thereof to produce signal 416. Signal 416 may be a single signal, or may be multiple signals transmitted over a single pathway or multiple pathways.

Pre-processor 420 may apply one or more signal processing operations to the signal generated by sensor 418. For example, pre-processor 420 may apply a pre-determined set of processing operations to the signal provided by sensor 418 to produce input signal 416 that can be appropriately interpreted by processor 412, such as performing A/D conversion. In some embodiments, A/D conversion may be performed by processor 412. Pre-processor 420 may also perform any of the following operations on the signal provided by sensor 418: reshaping the signal for transmission, multiplexing the signal, modulating the signal onto carrier signals, compressing the signal, encoding the signal, and filtering the signal. In some embodiments, pre-processor 420 may include a current-to-voltage converter (e.g., to convert a photocurrent into a voltage), an amplifier, a filter, and A/D converter, a de-multiplexer, any other suitable pre-processing components, or any combination thereof. In some embodiments, pre-processor 420 may include one or more components from front end processing circuitry 150 of FIG. 1.

In some embodiments, signal 416 may include PPG signals corresponding to one or more light frequencies, such as an IR PPG signal and a Red PPG signal. In some embodiments, signal 416 may include signals measured at one or more sites on a subject's body, for example, a subject's finger, toe, ear, arm, or any other body site. In some embodiments, signal 416 may include multiple types of signals (e.g., one or more of an ECG signal, an EEG signal, an acoustic signal, an optical signal, a signal representing a blood pressure, and a signal representing a heart rate). Signal 416 may be any suitable biosignal or any other suitable signal.

In some embodiments, signal 416 may be coupled to processor 412. Processor 412 may be any suitable software, firmware, hardware, or combination thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may, for example, include an assembly of analog electronic components. Processor 412 may calculate physiological information. For example, processor 412 may compute one or more of blood oxygen saturation, pulse rate, respiration rate, blood pressure, or any other suitable physiological parameter. Processor 412 may perform any suitable signal processing of signal 416 to filter signal 416, such as any suitable band-pass filtering, adaptive filtering, closed-loop filtering, any other suitable filtering, and/or any combination thereof. Processor 412 may also receive input signals from additional sources (not shown). For example, processor 412 may receive an input signal containing information about treatments provided to the subject. Additional input signals may be used by processor 412 in any of the calculations or operations it performs in accordance with processing system 400.

In some embodiments, all or some of pre-processor 420, processor 412, or both, may be referred to collectively as processing equipment.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store fiducial information or initialization information corresponding to physiological monitoring. In some embodiments, processor 412 may store physiological measurements or previously received data from signal 416 in a memory device for later retrieval. In some embodiments, processor 412 may store calculated values, such as a pulse rate, a blood pressure, a blood oxygen saturation, a fiducial point location or characteristic, an initialization parameter, or any other calculated values, in a memory device for later retrieval.

Processor 412 may be coupled to output 414. Output 414 may be any suitable output device such as one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into physiological monitoring system 100 of FIG. 1 in which, for example, input signal generator 410 may be implemented as part of sensor 102, or into physiological monitoring system 310 of FIG. 3 in which, for example, input signal generator 410 may be implemented as part of sensor unit 312 of FIG. 3, and processor 412 may be implemented as part of monitor 104 of FIG. 1 or as part of monitor 314 of FIG. 3. Furthermore, all or part of system 400 may be embedded in a small, compact object carried with or attached to the subject (e.g., a watch, other piece of jewelry, or a smart phone). In some embodiments, a wireless transceiver (not shown) may also be included in system 400 to enable wireless communication with other components of physiological monitoring systems 100 of FIGS. 1 and 310 of FIG. 3. As such, physiological monitoring systems 100 of FIGS. 1 and 310 of FIG. 3 may be part of a fully portable and continuous subject monitoring solution. In some embodiments, a wireless transceiver (not shown) may also be included in system 400 to enable wireless communication with other components of physiological monitoring systems 100 of FIGS. 1 and 310 of FIG. 3. For example, pre-processor 420 may output signal 416 over BLUETOOTH, 802.11, WiFi, WiMax, cable, satellite, Infrared, or any other suitable transmission scheme. In some embodiments, a wireless transmission scheme may be used between any communicating components of system 400. In some embodiments, system 400 may include one or more communicatively coupled modules configured to perform particular tasks. In some embodiments, system 400 may be included as a module communicatively coupled to one or more other modules.

It will be understood that the components of signal processing system 400 that are shown and described as separate components are shown and described as such for illustrative purposes only. In other embodiments the functionality of some of the components may be combined in a single component. For example, the functionality of processor 412 and pre-processor 420 may combined in a single processor system. Additionally, the functionality of some of the components shown and described herein may be divided over multiple components. Additionally, signal processing system 400 may perform the functionality of other components not show in FIG. 4. For example, some or all of the functionality of control circuitry 110 of FIG. 1 may be performed in signal processing system 400. In other embodiments, the functionality of one or more of the components may not be required. In an embodiment, all of the components can be realized in processor circuitry.

In some embodiments, any of the processing components and/or circuits, or portions thereof, of FIGS. 1, 3, and 4 may be referred to collectively as processing equipment. For example, processing equipment may be configured to amplify, filter, sample and digitize input signal 416 (e.g., using an analog-to-digital converter), and calculate physiological information from the digitized signal. Processing equipment may be configured to generate light drive signals, amplify, filter, sample and digitize detector signals, and calculate physiological information from the digitized signal. In some embodiments, all or some of the components of the processing equipment may be referred to as a processing module.

FIG. 5 is a block diagram of optical monitoring system 500 in accordance with some embodiments of the present disclosure. System 500 includes light drive module 502, transmission module 504, LED module 506, tissue 508, detector module 510, receiving module 512, and processing module 514. In some embodiments, channel 518 includes transmission module 504, LED module 506, detector module 510, and receiving module 512, as indicated by the dotted line. It will be understood that this particular border of the channel is merely exemplary and that any suitable elements or modules may be included or excluded from the channel.

In some embodiments, system 500 includes some elements described in physiological monitoring system 100 of FIG. 1. Light drive module 502 may include any suitable hardware, software, or combination thereof, used to generate a light drive signal. For example, a light drive module 502 may include some or all of light drive circuitry 120 of FIG. 1. In some embodiments, light drive module 502 may generate a signal such as the light drive signal of FIG. 2A.

In some embodiments, transmission module 504 includes any suitable connections between the generation of a signal by light drive module 502 and LED module 506. For example, transmission may include connectors, metallic conductors, wireless communications, any other suitable technique for transmitting a signal to LED module 506, and any combination thereof. In some embodiments, transmission module 504 may include amplifiers, filters, any other suitable conditioning techniques, or any combination thereof. In some embodiments, transmission module 504 may introduce signal distortions, noise, and other undesired signal features. Undesired features may be caused by non-ideal signal conduction, RF interference, parasitic capacitances, any other suitable source, and any combination thereof.

LED module 506 may include some or all of light source 130 of FIG. 1. In some embodiments, LED module 506 receives an electrical signal from transmission module 504 and generates an optical signal based on the received signal. For example, LED module 506 may include one or more LEDs. In some embodiments, LED module 506 may introduce signal distortions and other undesired signal features due to, for example, non-ideality of the electrical to optical conversion of LEDs, light source turn-on and turn-off rates, temperature-dependent parameters of the light source, any other suitable source, or any combination thereof.

Tissue 508 may include any suitable test subject tissue. For example, in a finger clip oximeter sensor, tissue 508 may include the finger of a test subject such as a patient. Tissue 508 may also include a calibration or testing sample such as a block of plastic. In some embodiments, tissue 508 may be omitted, for example in a calibration measurement. In some embodiments, tissue 508 may be opaque to the light, for example, in a calibration measurement. In some embodiments, as shown, tissue 508 is not included in channel 518.

Detector module 510 may include some or all of detector 140 of FIG. 1. For example, in some embodiments, detector module 510 receives a portion of the light emitted from LED module 506 and converts the optical signal to an electrical signal.

Receiving module 512 includes any suitable communication and processing components that communicate the signal output by detector module 510 to processing module 514. In some embodiments, receiving module 512 may include signal transmission elements such as those described for transmission module 504. In some embodiments, receiving module 512 may include a high pass filter or other suitable filter configured to attenuate ambient light or other light not emitted by LED module 506 from the signal. In some embodiments, receiving module 512 may include some or all of front end processing circuitry 150 of FIG. 1. For example, receiving module 512 may include analog-to-digital converter 158 of FIG. 1. It will be understood that in some embodiments, receiving module 512 may include no elements of front end processing 150. In some embodiments, receiving module 512 may include all elements of the system prior to processing module 514, and thus any elements between the detector and processing module 514 may be included in channel 518.

Processing module 514 may include any suitable elements of front end processing 150 of FIG. 1, back end processing 170 of FIG. 1, any other suitable processing, or any combination thereof. In some embodiments, processing module 514 receives a signal from receiving module 512. In some embodiments, the received signal includes signal features associated with tissue 508 and signal features associated with channel 518.

As described above, channel 518 may include the elements within the dotted border of system 500. As illustrated, channel 518 may omit a test subject such as tissue 508. Channel 518 may introduce distortions, modifications, and other undesirable channel effects to a signal as it passes through elements of the channel. It will be understood that the distortions and other modifications associated with the channel may affect the signal in addition to the desired physiological sensitivity of the system. Modifications may result in signal droop, overshoot, undershoot, attenuation, ringing, high frequency distortion, rise and fall time distortion, other suitable modifications, or any combination thereof. For example, channel 518 may include ambient subtractor 162 of FIG. 1, which may introduce droop into a square wave signal. This may occur, for example, where ambient light subtraction is implemented using a high pass filter, and the high pass filter attenuates the low frequency components of a detector signal. In some embodiments, droop is observed in a square wave signal when the output voltage within one pulse slowly decreases. Overshoot is observed when the rising edge of a pulse initially exceeds the intended output voltage before settling to the intended voltage. Undershoot is observed when the rising edge of a pulse fails to initially reach the intended output voltage before settling to the intended voltage. Ringing is observed when a voltage output oscillates above and below an intended value. Rise and fall time distortion is observed when the system reacts slowly to the change on a rising or falling edge of a pulse, and the rise and fall times of a signal are increased. Signals may pass through channel 518 and result in a distorted output received by processing module 514. It will be understood that the aforementioned distortions are merely exemplary and that any suitable distortion may be introduced by the system.

It will be understood that the particular elements shown in system 500 as being included in channel 518 are merely exemplary and that the channel may include or exclude any suitable electrical components, optical components, processing components, transmission components, any other suitable components, or any combination thereof.

FIG. 6 shows an illustrative determination of pulse shaping, in accordance with some embodiments of the present disclosure. In some embodiments, the behavior of square input 602, channel 604, and distorted output 606 illustrates the behavior of the system without pulse shaping. The behavior of pre-shaped input 610, channel 612, and square output 614 illustrates the behavior of a shaped pulse input without a test subject. In some embodiments, processing block 608 determines the characteristics of the shaping based on distorted output 606, using iterative refinements from square output 614, using modeling of the components in the channel, based on any other suitable information, or any combination thereof. In some embodiments, channel 604 and/or channel 612 correspond to channel 518 of FIG. 5.

In some embodiments, square input 602, channel 604, and distorted output 606 illustrate the effects of signal channel characteristics on a signal when the input signal is not shaped. For example, a square input signal may be the drive signal used to drive LEDs in a pulse oximeter. Square input 602 may, for example, drive light source 130 of FIG. 1.

In some embodiments, the system may shape the input signal in such a way that the output is a square wave or has any other desired characteristics. In some embodiments, signal distortions may reduce the sensitivity, quality, or other metrics of the physiological parameters monitored by the system by distorting signals. In some embodiments, distortions are filtered from the received signal using, for example, an inverse high pass filter or a filter having inverse properties of the distortion. In some embodiments, pulse shaping an input has a relatively lower power consumption load than digitally filtering a received signal.

In some embodiments, pre-shaped input 610, channel 612, and square output 614 are used to iteratively refine pulse shaping parameters. For example, when shaping is applied to the input, an output may be received at square output 614 that has some of the unwanted channel effects removed, but is still not ideal. Processing 608 may iteratively refine the shaping parameters provided to pre-shaped input 610 until the output received at square output 614 is as desired. It will be understood that in an iterative process, pre-shaped input 610 may begin with no shaping. It will also be understood that an iterative process may use predetermined information such as a factory calibration as a starting point. It will also be understood that square output 614 may not reach ideal characteristics.

In some embodiments, updating of shaping parameters based on an output signal may occur at regular intervals, for example, once every 10 pulses, or once a minute. In some embodiments, a calibration step may be performed at startup where the signal is not exposed to subject tissue and thus the signal distortions are associated only with non-subject parameters. In some embodiments of calibration, a polytetrafluoroethylene (PTFE) or other relatively inert replacement is used for the subject tissue.

In an example, the system may iteratively alter an input signal based on an output signal until the output signal displays the desired characteristics. In another example, the components of the channel (e.g., signal filters) may be modeled and pulse shaping characteristics determined based on that model. In another example, pulse shaping characteristics may be based on a model and iteratively refined based on an output signal. In some embodiments, a calibration may be performed before using the sensor with a test subject, for example if the calibration element is including as part of the sensor packaging and the sensor is plugged in prior to use on a patient. In some embodiments, a calibration performed at any suitable time may be refined at any suitable time. For example, a manufacturing calibration may be refined once at system startup. In some embodiments, a calibration may be done during development or manufacturing and stored in memory (e.g., non-volatile memory) on the circuit board of a monitor and/or on other components such as memory on the cable or sensor, such that the pulse shaping required for each component can be determined from the memory on any suitable combination of sensors and cables and circuit boards in use at any given time. In some embodiments, calibration values may include alternate calibrations to use at different ambient temperatures or coefficients which are used in an equation to modify the calibration based on ambient temperature. Any suitable element may be used to determine one or more temperatures for calibration purposes or otherwise.

It will be understood that calibration at any suitable point may include calibration and/or training signals additionally or alternatively to the pulse shape used in normal operation by the system. For example, a training signal may include a range of frequency and time elements in order to simulate physiological operation. In another example, a training signal may include several particular data points in order to establish a calibration curve. Training signals may include sinusoids, noise signals, chirps, impulses, a pseudo-random sequence, any other suitable signal, or any combination thereof. For example, a white noise signal may be used to estimate a frequency response of the channel.

In some embodiments, the shaping may be configured based on an emitter and detector that bypass a test subject. The system may include at least a first light emitter directed towards a test subject while in operation, and a second emitter that bypasses the test subject and is received directly by the same or a different detector. The bypassed signal may be used to determine the channel response independent of the subject, and thus may be used to determine shaping parameters. The shaping parameters may be used to mitigate the channel response in the received physiological signal. In some embodiments, a received signal is collected over a long duration relative to physiological changes, and is used to determine a channel response and/or to refine shaping parameters. Over a relatively long time period, the test subject response may average out to zero or approximately zero.

It will be understood that the arrows in FIG. 6 connecting distorted output 606 to processing block 608 to pre-shaped input 610 are merely illustrative, may represent data flow, and may be rearranged, omitted, repeated, or otherwise modified based on, for example, iteration schemes of processing 608.

In some embodiments, the characteristics of channel 604 may in part depend on a cable assembly including cables, one or more emitters, and one or more detectors, that is connected to a monitor. In some embodiments, the monitor may include predetermined pulse shaping parameters that are used with a particular cable assembly. In some embodiments, the monitor may include a selectable list or menu including, for example, model numbers associated with cable assemblies. In some embodiments, the system may automatically detect a cable assembly (e.g., by interrogating the cable assembly or by any other suitable technique). In some embodiments, pulse shaping parameters are determined by modeling the components, by an iterative method, or any combination thereof.

Pre-shaped input 610 is an example of an input signal that includes shaping that is configured to negate, counteract, or otherwise attenuate the effects of channel 612 and thus result in square output 614 including the desired signal characteristics. In some embodiments, channel 612 may include the same or similar characteristics as channel 604. In an example, where channel 612 introduces droop into the signal, pulse-shaped input 610 may include a pulse shape that increases in output voltage over the duration of a pulse, as illustrated for pre-shaped input 610, such that when convolved with the channel characteristics, square output 614 includes the desired, square pulse shape.

It will be understood that the aforementioned techniques for pulse shaping are merely illustrative. It will also be understood that the pulse shaping may depend in part on the particular components of the channel, the monitor, the wavelengths of light used, the physiological parameter being monitored, any other suitable characteristics, or any combination thereof. It will also be understood that drive pulses, and corresponding pulse shaping, is not limited to square pulses and may be applied to any suitable waveform.

FIG. 7 shows illustrative signal chain 700 including a test subject in accordance with some embodiments of the present disclosure. In some embodiments, signal chain 700 illustrates some of the signals illustrated in FIG. 6 when a test subject is included.

Signal chain 700 includes pre-shaped input 702. In some embodiments, pre-shaped input 702 corresponds to pre-shaped input 610 of FIG. 6. In some embodiments, pre-shaped input 702 may be a light signal generated by light drive module 502 of FIG. 5. As described above, a shaped input may include any suitable shaping of any suitable waveform. For example, the waveform may include a square pulse, and it may be shaped to compensate for droop and/or phase shifts. It will be understood that the input signal may be any suitable signal. For example, signals may include square waves, sinusoidal waves, frequency division signals, any suitable time division multiplexing, any suitable frequency division multiplexing, or any combination thereof. It will also be understood that shaping may include any suitable modifications of a light drive pulse in any suitable domain.

The pre-shaped waveform from pre-shaped input 702 passes through channel 704. In some embodiments, channel 704 includes elements described for channel 518 of FIG. 5. In some embodiments, channel 704 is configured as described for channel 612 of FIG. 6.

After interacting with a portion of channel 704, the signal interacts with tissue 706. Tissue 706 may correspond to tissue 508 of FIG. 5. For example, tissue 706 may include a test subject such as a fingertip, earlobe, or other sensing location, a calibration sample, a calibration block of polytetrafluoroethylene (PTFE), any other suitable material or combination of materials, or any combination thereof. In some embodiments, tissue 706 includes physiological tissue.

In some embodiments, a signal interacts with the first part of channel 704, followed by interacting with tissue 706, followed by interacting with a section part of channel 704. In some embodiments, this corresponds to the signal path illustrated for channel 518 of FIG. 5 and tissue 508 of FIG. 5. It will be understood that the particular sequence illustrated in signal chain 700, and in FIG. 5, is merely exemplary and that the tissue and channel elements may be arranged and/or interspersed in any suitable order.

Output 708 includes the signal as it is output from the channel. For example, output 708 may correspond to the output of receiving module 512 of FIG. 5. In some embodiments, output 708 includes the pre-shaped input signal after it has interacted with the channel and the test subject. In some embodiments, output 706 will be relatively free of unwanted channel effects such as phase shifts, droop and rise distortions. In some embodiments, the peak height attenuations of output 708 correspond solely or primarily of physiological attenuations. In some embodiments, shaping removes unwanted channel effects that would be present in output 708 were shaping not used. In some embodiments, physiological information is determined based on output 708. In some embodiments, shaping improves the quality, accuracy, or other parameters of output 708, and accordingly improves physiological parameters determined based on output 708

FIG. 8 shows illustrative plot 800 of system signals in accordance with some embodiments of the present disclosure. In some embodiments, signals 802 and 804 are detector current signals as would be detected by light detector 140 of FIG. 1, following some or all of front end processing 150 of FIG. 1, or at any other suitable point. In some embodiments, the abscissa axis may be in units of time and the ordinate axis may be in amplitude units, for example, nanoamps. In some embodiments, signal 802 is a distorted output signal including droop. For example, signal 802 may correspond to distorted output 606 of FIG. 6. In some embodiments, the distortions of signal 802 may be the result of signal channel characteristics as described above, convolved with a square wave input signal. In some embodiments, signal 804 is a square output signal that is the result of using pulse shaping. For example, signal 804 may correspond to square output 614 of FIG. 6. It will be understood that some distortions are still present in signal 804. In some embodiments, these remaining distortions may include transient changes in channel characteristics, as pulse shaping may only correct for more predictable channel effects.

FIG. 9 is flow diagram 900 showing illustrative steps for shaping light drive pulses in accordance with some embodiments of the present disclosure.

In step 902, the system may determine a signal characteristic corresponding to a channel effect. In some embodiments, the channel corresponds to that described for channel 604 of FIG. 6. The channel characteristic may include the effect of channel components on a signal being transmitted by or through the channel. In some embodiments, the signal channel characteristic may include the aggregate frequency and/or amplitude response of the components in a channel such as channel 518 of FIG. 5. For example, the channel may include a drive pulse generator, a light emitter, a light detector, a high pass filter to attenuate ambient components, and an analog-to-digital converter. The channel characteristics may include all alterations and distortions to a signal introduced by those components, such that the output signal differs from the input signal. In some embodiments, the channel characteristic may include undesirable channel effects. In some embodiments, where the emitted light signal is attenuated by interacting with a subject's tissue before being detected, the channel characteristics may include all changes to the signal except for those introduced by the subject.

In step 904, the system may determine at least one light drive parameter based on the signal characteristic determined in step 902. In some embodiments, determining the light drive parameter includes determining a light drive shape for an output. Pulse shaping may include applying a light drive shape to a light drive signal, such as the signal from light drive module 502 of FIG. 5. The system may determine the light drive shape based on the signal characteristics determined in step 902. In some embodiments, the light drive shape may be an inverse or other mathematical conversion of the signal characteristics. In some embodiments, the determined light drive shape may include the result of convolving the channel characteristics with the desired pulse shape. In some embodiments, the light drive shape may be the inverse of that convolution. It will be understood that the aforementioned light drive shapes are merely exemplary and that any suitable light drive shape and any suitable technique for determining the light drive shape may be used.

In some embodiments, the system may generate the light drive parameter and generate a photonic signal using an emitter such as light emitter 130 of FIG. 1. In some embodiments, the received signal is used to determine physiological parameters. For example, the received signal may be used to determine blood oxygen saturation, blood pressure, pulse rate, respiration rate, any other suitable parameters, or any combination thereof.

In some embodiments, the light drive parameter is used to generate a light drive signal using a digital to analog converter. For example, a digital signal may be generated using any suitable processing equipment, and that digital signal may be converted to an analog light drive signal.

In some embodiments, light drive parameters may be determined, as described above, based on modeling of signal components, based on an output signal, by any other suitable technique, or any combination thereof. In some embodiments, the system may retrieve predetermined parameters that are stored, for example, in computer memory. In some embodiments, stored characteristics may be retrieved based on user input, based on automatic detection of attached components (e.g., a cable assembly), user selection of attached components from a menu, using any other suitable technique, or any combination thereof. In some embodiments, the system may determine characteristics in a calibration step, for example, before being connected to a subject. In some embodiments, the system may repeatedly determine signal characteristics while operating.

In some embodiments, modeling of signal components may include determining a model of the resistors, capacitors, inductors, amplifiers, cables, emitters, detectors, filters, parasitic contributions of included components, active components, passive components, any other suitable channel components, and any combination thereof. The impact of the components on a signal may be determined computationally, using test signals, using a calibration sample, based on individual components, based on collections of components, by any other suitable technique, or any combination thereof.

In some embodiments, the system may determine a light drive parameter by receiving a light signal, analyzing the light signal, and determining a channel characteristic based on the analysis. For example, where the input signal is a square wave and the output signal includes a particular amount of droop, the system may detect that droop as corresponding to a signal characteristic. In some embodiments, this determining may be an iterative process, where the light drive parameters are refined through successive iterations.

In some embodiments, predetermined channel characteristics may be updated based on a received light signal. For example, the system may use predetermined characteristics associated with a particular cable assembly, and may refine those characteristics based on a received signal.

It will be understood that the aforementioned techniques for determining signal characteristics and/or determining a light drive parameter are merely exemplary and that any suitable techniques, including those mentioned herein, may be used in any suitable combination. It will also be understood that while the examples described herein relate to light drive signals, shaping may be used with any suitable signals.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method of compensating for a channel effect in a physiological monitor, the method comprising:
determining, using processing equipment, a signal characteristic corresponding to a channel effect of a channel used to receive a light signal attenuated by a subject; and
determining, using the processing equipment, at least one light drive parameter, wherein the light drive parameter is based on the signal characteristic, wherein the at least one light drive parameter is configured to compensate for the channel effect, and wherein the at least one light drive parameter comprises light drive shape.

2. The method of claim 1, wherein determining the signal characteristic comprises retrieving data associated with signal characteristics, and wherein the data is based on modeled signal processing components.

3. The method of claim 2, wherein retrieving data associated with signal characteristics comprises automatically retrieving data based on the presence of components coupled to the monitor.

4. The method of claim 1, wherein determining the signal characteristic comprises:
receiving the light signal;
analyzing the light signal; and
determining, based on the analysis, the at least one light drive parameter.

5. The method of claim 1, wherein determining the signal characteristic comprises using an emitter and detector that bypass the subject.

6. The method of claim 1, wherein the at least one light drive parameter is used to generate a light drive signal for activating a light source to emit a photonic signal, and wherein the light drive signal is based on the at least one light drive parameter.

7. The method of claim 6, wherein the signal characteristic corresponds to a high pass filter effect of the channel, and wherein the light drive parameter is determined based on an inverse high pass filter.

8. The method of claim 1, further comprising:
receiving, using the processing equipment, the light signal attenuated by the subject; and
determining, using the processing equipment, a physiological parameter based on the light signal.

9. The method of claim 8, wherein determining a physiological parameter comprises determining a parameter selected from the group consisting of blood oxygen saturation, blood pressure, heart rate, respiration rate, respiration effort, and any combination thereof.

10. The method of claim 1, wherein the signal characteristic is a characteristic selected from the group consisting of a droop, rise time, fall time, overshoot, undershoot, ringing, phase shifts, and any combination thereof.

11. The method of claim 1, wherein the physiological monitor is a photoplethysmograph.

12. A system for compensating for a channel effect in a physiological monitor, the system comprising:
a sensor configured to receive a light signal attenuated by a subject; and
processing equipment configured to perform operations comprising:
determining a signal characteristic corresponding to a channel effect of a channel used to receive the light signal attenuated by the subject; and
determining at least one light drive parameter, wherein the light drive parameter is based on the signal characteristic, wherein the at least one light drive parameter is configured to compensate for the channel effect, and wherein the at least one light drive parameter comprises light drive shape.

13. The system of claim 12, wherein determining the signal characteristic comprises retrieving data associated with signal characteristics, and wherein the data is based on modeled signal processing components.

14. The system of claim 13, wherein retrieving data associated with signal characteristics comprises automatically retrieving data based on the presence of components coupled to the monitor.

15. The system of claim 12, wherein determining the signal characteristic comprises:
receiving the light signal;
analyzing the light signal; and
determining, based on the analysis, the at least one light drive parameter.

16. The system of claim 12, wherein determining the signal characteristic comprises using an emitter and detector that bypass the subject.

17. The system of claim 12, wherein the at least one light drive parameter is used to generate a light drive signal for activating a light source to emit a photonic signal, and wherein the light drive signal is based on the at least one light drive parameter.

18. The system of claim 17, wherein the signal characteristic corresponds to a high pass filter effect of the channel, and wherein the light drive parameter is determined based on an inverse high pass filter.

19. The system of claim 12, wherein the processing equipment is configured to perform operations further comprising:
receiving the light signal attenuated by the subject; and
determining a physiological parameter based on the light signal.

20. The system of claim 19, wherein determining a physiological parameter comprises determining a parameter selected from the group consisting of blood oxygen saturation, blood pressure, heart rate, respiration rate, respiration effort, and any combination thereof.

21. The system of claim 12, wherein the signal characteristic is a characteristic selected from the group consisting of a droop, rise time, fall time, overshoot, undershoot, ringing, phase shifts, and any combination thereof.

22. The system of claim 12, wherein the physiological monitor is a photoplethysmograph.
